(19) United States
(12) Patent Application Publication
(10) Pub. No.: US 2005/0142596 A1
(43) Pub. Date: Jun. 30, 2005

(54) Methods of diagnosing renal and cardiovascular disease

(76) Inventors: Andrzej S. Krolewski, Needham, MA (US); Grzegorz Placha, Jamaica Plain, MA (US)

Correspondence Address: FISH & RICHARDSON PC, 225 FRANKLIN ST, BOSTON, MA, 02110, US

(21) Appl. No.: 10/987,981

(22) Filed: Nov. 12, 2004

Related U.S. Application Data
(60) Provisional application No. 60/520,118, filed on Nov. 14, 2003.

(51) Int. Cl. C12Q 1/68

(52) U.S. Cl. 435/6

(57) ABSTRACT

The present invention provides methods for predicting risk of developing renal disease or coronary artery disease in a subject, by evaluating expression, levels, or activity of p21, or the presence or absence of polymorphic variants thereof. Also described are methods of treating or preventing renal or coronary artery disease.

### CLAIM OF PRIORITY

This application claims the benefit under 35 USC § 119(e) of U.S. Patent Application Ser. No. 60/520,118, filed on Nov. 14, 2003, the entire contents of which are hereby incorporated by reference.

### BACKGROUND

p21, also known as CIP1/WAF1, is an 18 kD protein (164 amino acids) that is an important intermediate by which p53 mediates its role as an inhibitor of cellular proliferation in response to DNA damage. p21 may bind to and inhibit cyclin-dependent kinase activity, preventing the phosphorylation of critical cyclin-dependent kinase substrates and blocking cell cycle progression, and thus proliferation. p21 is expressed in all adult human tissues.

In a streptozotocin model of diabetic nephropathy, p21 knock-out mice do not develop glomerular hypertrophy and proteinuria despite elevation of TGF-β1 IMRNA (Al-Douahji et al., Kidney Int. 56(5):1691-9 (1999).

### SUMMARY OF THE INVENTION

The invention is based, at least in part, on the inventors' discovery of a number of polymorphisms in the p21 gene (e.g., in the p21 promoter) that are statistically correlated with differential risk, e.g., decreased risk, for renal disease (e.g., diabetes related renal disease, such as nephropathy or end stage renal disease (ESRD)), coronary artery disease and/or mortality.

Accordingly, in one aspect, the invention features methods of evaluating a subject, preferably a human, e.g., determining a subject's risk of developing renal disease, e.g., diabetes-related renal disease, e.g., nephropathy, or end stage renal disease. The methods include evaluating a polymorphism, e.g., detecting a single nucleotide polymorphism (SNP), in a p21 gene of the subject, e.g., in a p21 gene, e.g., in the p21 promoter region. In a preferred embodiment, the methods include determining, for one or both alleles of a p21 gene of the subject, one or more of the following:
(a) the identity of the nucleotide at −2266 relative to the transcription start site of the p21 gene, NCBI name: rs4135234, also referred to herein as G−2266A. This is the nucleotide corresponding to position 98 of SEQ ID NO: 1, and position 942 of SEQ ID NO:4;
(b) the identity of the nucleotide at −1021 relative to the transcription start site of the p21 gene, NCBI name: rs762623, also referred to herein as G−1021A. This nucleotide corresponds to position 98 of SEQ ID NO: 2, and position 2188 of SEQ ID NO:4; and
(c) the identity of the nucleotide at +1004 relative to the transcription start site of the p21 gene, NCBI name: rs3176330, also referred to herein as G+1004A. This nucleotide corresponds to position 103 of SEQ ID NO: 3, and position 4212 of SEQ ID NO:4.

Relevant portions of the p21 gene are included in Genbank Accession no. Z85996.1. The presence of an adenine (A) at the position corresponding to position 98 of SEQ ID NO: 1, position 98 of SEQ ID NO: 2, and/or position 103 of SEQ ID NO: 3, in one or both alleles of the p21 gene of the subject, is correlated with a significantly decreased risk of developing renal disease, e.g., nephropathy or ESRD, and/or decreased risk of progressing to ESRD from proteinuria compared to a reference value, e.g., a value for the comparable risk for a subject carrying a guanine (G) allele in one or both chromosomes at the position corresponding to position 98 of SEQ ID NO: 1, position 98 of SEQ ID NO:2, and/or position 103 of SEQ ID NO:3. In humans, this is on chromosome 6.

In some embodiments, the determining step includes: obtaining a biological sample from the subject comprising a p21 gene or fragment thereof, and detecting one or more of: (a) the identity of the nucleotide corresponding to position 98 of SEQ ID NO: 1, (b) the identity of the nucleotide corresponding to position 98 of SEQ ID NO:2, and (c) the identity of the nucleotide corresponding to position 103 of SEQ ID NO:3 in a nucleic acid sample of the subject. The detection can be performed by any method known in the art, e.g., by one or more of: chain termination sequencing, restriction digestion, allele-specific polymerase reaction, single-stranded conformational polymorphism analysis, genetic bit analysis, temperature gradient gel electrophoresis, ligase chain reaction, or ligase/polymerase genetic bit analysis, allele specific hybridization, size analysis, nucleotide sequencing, 5′ nuclease digestion, primer specific extension, or oligonucleotide ligation assay.

In some embodiments, the methods include diagnosing a subject as being at risk for or having renal disorder, e.g., a renal disorder described herein. In another embodiment, the method includes prescribing or beginning a treatment for renal disease in the subject, e.g., administering an ACE inhibitor. In some embodiments, the methods include performing a second diagnostic test, e.g., evaluating one or more of: insulin metabolism, plasma glucose levels, urine protein levels, and glomerular filtration rate.

A subject is typically a human, e.g., a human with diabetes, overt proteinuria, and/or a family history of renal disease or diabetes. The biological sample can be a cell sample, tissue sample, or at least partially isolated molecules, e.g., nucleic acids, e.g., genomic DNA, cDNA, mRNA, and/or proteins derived from the subject. Such methods are useful, e.g., for diagnosis of a renal disorder, e.g., a diabetes related renal disorder, e.g., nephropathy or end stage renal disease. A biological sample from the subject is a sample including at least one cell, e.g., a blood sample or an epithelial cell sample, e.g., a cheek cell sample.

In one embodiment, detecting a mutation or polymorphism can include: (i) providing a probe or primer, e.g., a labeled probe or primer, that includes a region of nucleotide sequence that hybridizes to a sense or antisense sequence from a p21 gene (e.g., from a p21 promoter) or naturally occurring mutants thereof, or to the 5′ or 3′ flanking sequences naturally associated with a p21 gene; (ii) exposing the probe/primer to nucleic acid of the subject; and detecting, e.g., by hybridization, e.g., in situ hybridization to the nucleic acid; or amplification of the nucleic acid, the presence or absence of the mutation or polymorphism, e.g., a polymorphism shown in FIG. 1.

In a preferred embodiment, the methods include performing one or more of the following determinations, for one or both chromosomes of the subject:
(a) determining the identity of the nucleotide of the p21 gene corresponding to position 98 of SEQ ID NO: 1, e.g., determining whether either the coding or non coding strand of a p21 gene of the subject includes the nucleotide sequence of SEQ ID NO: 1 having a polymorphism at nucleotide 98, e.g., determining if the coding or non coding strand of a p21 gene of the subject includes the nucleotide sequence of SEQ ID NO: 1 where position 98 is an A;
(b) determining the identity of the nucleotide of the p21 gene corresponding to position 98 of SEQ ID NO: 2, e.g., determining whether either the coding or non coding strand of a p21 gene of the subject includes the nucleotide sequence of SEQ ID NO:2 having a polymorphism at nucleotide 98, e.g., determining if the coding or non coding strand of a p21 gene of the subject includes the nucleotide sequence of SEQ ID NO: 1 where nucleotide 98 is an A; and/or
(c) determining the identity of the nucleotide of the p21 gene corresponding to position 103 of SEQ ID NO: 3, e.g., determining whether either the coding or non coding strand of a p21 gene of the subject includes the nucleotide sequence of SEQ ID NO:3 having a polymorphism at nucleotide 103, e.g., determining if the coding or non coding strand of a p21 gene of the subject includes the nucleotide sequence of SEQ ID NO:3 where nucleotide 103 is an A.

In some embodiments, the determining step includes amplifying at least a portion of a p21 nucleic acid molecule of the subject, e.g., a portion of the p21 promoter, e.g., a portion including a polymorphism described herein.

In some embodiments, the determining step includes sequencing at least a portion of a p21 nucleic acid molecule of the subject, e.g., a portion of the promoter, e.g., a portion including a polymorphism described herein.

In some embodiments, the determining step includes hybridizing a p21 nucleic acid molecule of the subject with a probe or primer, e.g., a probe or primer described herein.

In another embodiment, the methods include determining the activity of, or the presence or absence of, p21 nucleic acid molecules and/or polypeptides or in a biological sample.

In one embodiment, the methods include generating a dataset of the result of the determination, e.g., generating print or computer readable material, e.g., an informational, diagnostic, marketing or instructional print material or computer readable medium. The material can include information correlating the result of the determination with the subject's risk of developing renal disease, e.g., diabetes related renal disease, e.g., nephropathy, or end stage renal disease. The methods can include providing the print or computer readable material to the subject or to the health care provider.

Methods of the invention can be used prenatally or to determine if a subject's offspring will be at risk for a disorder.

In another aspect, the invention features an isolated nucleic acid, e.g., a probe or primer, or partial or complete cDNA, or a genomic fragment, or its complement, wherein the nucleic acid includes at least 10, e.g., at least 15, 20, 25, 30, 35 or more contiguous nucleotides of any one of SEQ ID NOs:1, 2, 3, or 4. In some embodiments, the nucleic acid includes at least 10, e.g., at least 15, 20, 25, 30, 35 or more contiguous nucleotides of any one of:
(a) SEQ ID NO:1, wherein the nucleic acid includes nucleotide 98 of SEQ ID NO:1;
(b) SEQ ID NO:2, wherein the nucleic acid includes nucleotide 98 of SEQ ID NO:2; and/or
(c) SEQ ID NO:3, wherein the nucleic acid includes nucleotide 103 of SEQ ID NO:3.

In some embodiments, the isolated nucleic acid or its complement includes a detectable label, e.g., a radiolabel, fluorescent label, bioluminescent label, chemiluminescent label, nucleic acid, hapten, enzyme label, or colorimetric label.

In some embodiments, the nucleic acid or its complement includes less than 200 contiguous nucleotides, e.g., less than 150, 100, or contiguous nucleotides of the subject sequence.

In some embodiments, the probe or primer is 500 nucleotides or less in length, e.g., about, 400, 300, 250, 200, or 100 nucleotides or less in length.

In one embodiment, the nucleic acid, or its complement, is attached to a solid support, e.g., the nucleic acid is part of an array of nucleic acids, e.g., an array that includes one, 2, 3 or more of the nucleic acids of (a)-(c) described above.

In one embodiment, the nucleic acid, or its complement, hybridizes specifically to the sequence of SEQ ID NO:1 where position 98 is a G but not to the sequence of SEQ ID NO:1 where position 98 is an A.

In one embodiment, the nucleic acid, or its complement, hybridizes specifically to the sequence of SEQ ID NO: 1 where position 98 is an A but not to the sequence of SEQ ID NO: 1 where position 98 is a G.

In one embodiment, the nucleic acid, or its complement, hybridizes specifically to the sequence of SEQ ID NO:2 where position 98 is a G but not to the sequence of SEQ ID NO:2 where position 98 is an A.

In one embodiment, the nucleic acid, or its complement, hybridizes specifically to the sequence of SEQ ID NO:2 where position 98 is an A but not to the sequence of SEQ ID NO:2 where position 98 is a G.

In one embodiment, the nucleic acid, or its complement, hybridizes specifically to the sequence of SEQ ID NO:3 where position 103 is a G but not to the sequence of SEQ ID NO:3 where position 103 is an A.

In one embodiment, the nucleic acid, or its complement, hybridizes specifically to the sequence of SEQ ID NO:3 where position 103 is an A but not to the sequence of SEQ ID NO:3 where position 103 is a G.

In some embodiments, hybridization of the probe or primer allows determination of the identity of the nucleotide at one or more of (a) the nucleotide corresponding to position 98 of SEQ ID NO: 1, (b) the nucleotide corresponding to position 98 of SEQ ID NO: 2, and (c) the nucleotide corresponding to position 103 of SEQ ID NO: 3. In some embodiments, the probe or primer hybridizes adjacent to, e.g., within about 25, 50, 100, 500, 1000, 5000, or 10,000 nucleotides of, one or more of (a) the nucleotide corresponding to position 98 of SEQ ID NO: 1, (b) the nucleotide corresponding to position 98 of SEQ ID NO: 2, and (c) the nucleotide corresponding to position 103 of SEQ ID NO: 3.

In some embodiments, the nucleic acid hybridizes to a restriction fragment, wherein one or more of (a) the nucleotide corresponding to position 98 of SEQ ID NO: 1, (b) the nucleotide corresponding to position 98 of SEQ ID NO: 2, and (c) the nucleotide corresponding to position 103 of SEQ ID NO: 3 forms part of a restriction enzyme recognition site at one end of the fragment. In some embodiments, the site is recognized by a restriction enzyme selected from the group consisting of HaeI, BaII, and CviJI.

In another aspect, the invention features an array of nucleic acid molecules, e.g., nucleic acid molecules attached to a solid support. The array includes 2 or more p21 nucleic acids, e.g., probes or primers described herein, that are capable of detecting (e.g., hybridizing to) a p21 polymorphism, e.g., a p21 polymorphism described herein. For example, the array can include one, 2, 3 or more of the probes or primers described herein.

In another aspect, the invention features a set (i.e., a plurality) of oligonucleotides, e.g., primers, for amplifying a genomic sequence that spans a p21 polymorphism, e.g., a polymorphism in the p21 promoter, e.g., a p21 polymorphism described herein. FIG. 1 shows numerous p21 polymorphisms associated with a renal disorder, e.g., a renal disorder described herein, in the context of the surrounding genomic sequence. One of skill in the art could easily design a set of primers to amplify any one or more of the polymorphisms described herein. For example, the set can include a plurality of oligonucleotides, each of which is at least partially complementary (e.g., at least 50%, 60%, 70%, 80%, 90%, 92%, 95%, 97%, 98%, 99%, or 100% complementary) to a p21 nucleic acid, e.g., a p21 nucleic acid described herein.

In one embodiment, the set includes two or more probes or primers described herein.

In one embodiment the set includes a first and a second oligonucleotide. The first and second oligonucleotide can hybridize to the same or to different locations of SEQ ID NO:1, 2, 3 or 4, or the complement of SEQ ID NO:1, 2, 3 or 4. Different locations can be different but overlapping, or non-overlapping on the same strand. The first and second oligonucleotide can hybridize to sites on the same or on different strands. The set can be useful, e.g., for identifying SNPs, or identifying specific polymorphisms or alleles of p21. In some embodiments, each oligonucleotide of the set has a different nucleotide at an interrogation position. In some embodiments, the set includes two oligonucleotides, each complementary to a different allele at a locus, e.g., a biallelic or polymorphic locus.

In another embodiment, the set includes at least four oligonucleotides, each having a different nucleotide (e.g., adenine, guanine, cytosine, or thymidine) at the interrogation position (i.e., position 98 of SEQ ID NO:1, position 98 of SEQ ID NO:2, and/or position 103 of SEQ ID NO:3). The interrogation position can be a SNP or the site of a mutation. In another preferred embodiment, the oligonucleotides of the set are identical in sequence to one another, except for differences in length. The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has at least one nucleotide change at a position in addition to a query position, e.g., a destabilizing mutation to decrease the Tm of the oligonucleotide. In another embodiment, at least one oligonucleotide of the set has at least one non-natural nucleotide, e.g., inosine. In some embodiments, the oligonucleotides are attached to a solid support, e.g., to different addresses of an array or to different beads or nanoparticles.

In some embodiments the set of oligonucleotides can be used to specifically amplify (e.g., by PCR), and/or detect, a p21 nucleic acid comprising a polymorphism described herein.

The set described herein can be part of a kit including at least one probe nucleic acid or antibody reagent described herein, and instructions for using the kit to evaluate susceptibility for a renal disorder in a subject. The kit can be used, e.g., by a subject or health care provider.

In another aspect, the invention features methods of evaluating, e.g., diagnosing, a subject. The methods include identifying a subject suspected of being at risk for, e.g., a subject having a family history of, a renal disorder, e.g., a renal disorder described herein, or an associated condition, e.g., diabetes. The methods typically include: providing a nucleic acid sample from the subject; evaluating a genotype of the p21 gene of the subject, e.g., evaluating the presence or absence of a polymorphism described herein in the subject's p21 gene, e.g., the presence or absence of a p21 polymorphism described herein (e.g., by determining the identity or sequence of at least a portion of a p21 allele); and comparing the genotype, e.g., the haplotype, of the subject's p21 gene to a reference. The method optionally includes providing a treatment for the renal disorder to the subject.

Because the p21 polymorphisms described herein (e.g., the G to A SNPs described herein) are also correlated with decreased risk of coronary artery disease (CAD) and increased age, any of the methods and compositions described herein for the evaluation of risk of renal disease can also be used to evaluate risk of developing CAD or of early mortality compared to a reference value, e.g., compared to the risk for a control subject.

In another aspect, the invention features methods of identifying agents for the treatment of renal disease or coronary artery disease. The methods include: identifying an agent that modulates, e.g., decreases, p21 expression, levels or activity (e.g., permanently or temporarily); and correlating the ability of an agent to decrease p21 expression, levels or activity with the ability to treat renal disease or CAD. In one embodiment, the ability of the agent to interact with, e.g., to bind, p21 is evaluated. In another embodiment, the effect of the agent to interact with a p21 regulatory region, e.g., a p21 promoter, is evaluated.

In some embodiments, the invention includes methods for identifying candidate compounds for treatment of renal disease or coronary artery disease (CAD). The methods include providing a sample comprising a p21 polypeptide or nucleic acid; contacting the sample with a test compound; and evaluating an effect of the test compound on a level, expression, or activity of the p21 nucleic acid or polypeptide. A test compound that decreases a level, expression, or activity of the p21 nucleic acid or polypeptide is a candidate compound for treatment of renal disease or CAD. Further, the invention includes methods for identifying candidate therapeutic agents for treatment of renal disease or coronary artery disease (CAD). The methods include providing an animal model of renal disease or CAD; contacting the animal model with a candidate compound that decreases a level, expression, or activity of the p21 nucleic acid or polypeptide; and evaluating an effect of the candidate compound on a parameter of the disease in the animal model; wherein a candidate compound that improves a parameter of the disease is a candidate therapeutic agent for treatment of renal disease or CAD. The methods can further include administering the candidate therapeutic agent to a subject, e.g., a subject in a clinical trial having renal disease or CAD, and evaluating an effect of the candidate therapeutic agent on the disease in the subject.

The method can include correlating the effect of the agent on p21 with a predicted effect of the agent on a mammal, e.g., a human, e.g., by providing (e.g., to the government, a health care provider, insurance company or patient) informational, marketing or instructional material, e.g., print material or computer readable material (e.g., a label or email), related to the agent or its use, identifying the agent as a possible or predicted treatment in a mammal, e.g., a human. The methods can include identifying the agent as a treatment or lead compound for treatment or prevention of renal disease or CAD, e.g., in humans, if it decreases p21 expression, levels or activity. The identification can be in the form of informational, marketing or instructional material, e.g., as described herein. In one embodiment, the method includes correlating a value for decreased p21 expression with an ability to treat renal disease or CAD, e.g., generating a dataset of the correlation.

In some embodiments, the method includes evaluating, e.g., quantitatively or qualitatively measuring, the effect of the agent on cardiovascular tissue, e.g., evaluating one or more of renal function or cardiac electrical activity, e.g., heart contractility, heart rate, or ventricular function (of a subject). Evaluating the effect of the agent on cardiovascular function or development can include administering the agent to an experimental mammal, to a renal or cardiovascular tissue of the animal, e.g., an animal model for renal disease or CAD. In some embodiments, the evaluation includes entering a value for the evaluation, e.g., into a database or other record.

In a preferred embodiment, the subject is an experimental animal. The animal can be wild-type or a transgenic experimental animal, e.g., a p21 transgenic or knockout rodent, e.g., p21-overexpressing mouse. The subject can also be a human. In a preferred embodiment, the evaluating step comprises administering the agent to the subject and evaluating a parameter of cardiovascular function.

In a preferred embodiment, the identifying step includes: (a) providing an agent to a cell, tissue or non-human animal whose genome includes an exogenous nucleic acid that includes a regulatory region of p21, e.g., a p21 promoter, operably linked to a nucleotide sequence encoding a reporter polypeptide (e.g., a light based, e.g., a calorimetric (e.g., LacZ) or fluorescently detectable label, e.g., a fluorescent reporter polypeptide, e.g., green fluorescent protein (GFP), blue fluorescent protein (BFP), red fluorescent protein (RFP), yellow fluorescent protein (YFP), or an enhanced revision thereof, e.g., enhanced GFP (EGFP); (b) evaluating the ability of a test agent to modulate the expression of the reporter polypeptide in the cell, tissue or non-human animal; and (c) selecting a test agent that modulates the expression of the reporter polypeptide as an agent that modulates p21. In one embodiment, the cell or tissue is a renal or cardiovascular cell or tissue, e.g., a renal cell or myocyte. In another embodiment, the non-human animal is a transgenic animal, e.g., a transgenic rodent, e.g., a mouse, rat or guinea pig, harboring the nucleic acid. In yet another embodiment, a cell, e.g., renal cell or myocyte, is derived from a transgenic animal.

The test agent can be, e.g., a nucleic acid (e.g., an antisense, SiRNA, or ribozyme), a polypeptide (e.g., an antibody or antigen-binding fragment thereof), a peptide fragment, a peptidomimetic, or a small molecule (e.g., a small organic molecule with a molecular weight of less than 2000 daltons). In one embodiment, the test agent is a member of a combinatorial library, e.g., a peptide or organic combinatorial library, or a natural product library. In some embodiments, a plurality of test agents, e.g., library members, is tested. In some embodiments, the test agents of the plurality share structural or functional characteristics. Test agents can also be crude or semi-purified extracts, e.g., a botanical extract such as a plant extract, or an algal extract.

In one embodiment, the methods include two evaluating steps, e.g., the method includes a first step of evaluating the test agent in a first system, e.g., a cell or tissue system, and a second step of evaluating the test agent in a second system, e.g., a second cell or tissue system or in a non-human animal. In other embodiments, the methods include two evaluating steps in the same type of system, e.g., the agent is re-evaluated in a non-human animal after a first evaluation in the same or a different non-human animal. The two evaluations can be separated by any length of time, e.g., days, weeks, months or years. In some embodiments, the methods include optimizing the test agent.

In another aspect, the invention features methods of evaluating a subject, e.g., determining if a subject is at risk for renal disease or CAD. The methods include evaluating the gene structure, expression, protein level or activity of p21 in the subject. The method includes (a) evaluating the level, activity, expression and/or genotype of a p21 molecule in a subject, e.g., in a biological sample from the subject, and (b) correlating an alteration in a p21 molecule, e.g., a less than wild-type level, activity, expression, and/or a mutation of p21 with a decreased risk for renal disease or CAD. Correlating means identifying the alteration as a risk or diagnostic factor for renal disease or CAD, e.g., providing a print material or computer readable medium, e.g., an informational, diagnostic, marketing or instructional print material or computer readable medium, e.g., to the subject or to a health care provider, identifying the alteration as a risk or diagnostic factor for renal disease or CAD.

In one embodiment, the methods include diagnosing a subject as being at risk for or having renal disease or CAD. In one embodiment, the methods include prescribing or beginning a treatment for renal disease or CAD. In some embodiments, the methods include performing a second diagnostic test, e.g., evaluating one or more of renal function or cardiac electrical activity, e.g., heart contractility, heart rate, or ventricular function (of a subject).

The subject is typically a human, e.g., a human with a family history of renal disease or CAD. The biological sample can be a cell sample, tissue sample, or at least partially isolated molecules, e.g., nucleic acids, e.g., genomic DNA, cDNA, mRNA, and/or proteins derived from the subject. Such methods are useful, e.g., for diagnosis of renal disease or CAD.

In some embodiments, the methods include one or more of the following:
1) detecting, in a biological sample from the subject, the presence or absence of a mutation (e.g., a polymorphism as described herein) that affects the expression of p21, or detecting the presence or absence of a mutation in a region that controls the expression of the p21 gene, e.g., a mutation in the 5′ control region, the presence of a mutation being indicative of decreased risk;
2) detecting, in a biological sample from the subject, the presence or absence of a mutation that alters the structure of p21, the presence of a mutation being indicative of decreased risk;
3) detecting, in a biological sample from the subject, the misexpression of p21, at the mRNA level, e.g., detecting a non-wild-type level of a p21 mRNA, decreased levels of p21 mRNA being associated with decreased risk. Detecting misexpression can include ascertaining the existence of at least one of: an alteration in the level of a mRNA transcript of p21 compared to a reference, e.g., as compared to a baseline value or to levels in a subject not at risk for renal disease or CAD; the presence of a non-wild-type splicing pattern of a mRNA transcript of the gene; or a non-wild-type level of p21 protein e.g., as compared to a reference, e.g., compared to a baseline value, or to levels in a subject not at risk for renal disease or CAD;
4) detecting, in a biological sample from the subject, the misexpression of p21, at the protein level, e.g., detecting a non-wildtype level of a p21 polypeptide, decreased levels of p21 protein (e.g., compared to a control) being indicative of a decreased risk. For example, the method can include contacting a sample from the subject with an antibody to p21 protein; and/or
5) detecting, in a biological sample from the subject, a polymorphism, e.g., a SNP, in p21, e.g., a SNP described herein. In some embodiments the methods include: ascertaining the existence of at least one of: an insertion or a deletion of one or more nucleotides from p21; a point mutation, e.g., a substitution of one or more nucleotides of the gene; and/or a gross chromosomal rearrangement of the gene, e.g., a translocation, inversion, duplication or deletion. In one embodiment, a SNP or haplotype described herein is detected.

In one embodiment, detecting a mutation or polymorphism can include: (i) providing a probe or primer, e.g., a labeled probe or primer, that includes a region of nucleotide sequence that hybridizes to a sense or antisense sequence from p21, or naturally occurring mutants thereof, or to the 5′ or 3′ flanking sequences naturally associated with p21; (ii) exposing the probe/primer to nucleic acid of the subject; and (iii) detecting, e.g., by hybridization, e.g., in situ hybridization to the nucleic acid; or amplification of the nucleic acid, the presence or absence of the mutation or polymorphism.

In one embodiment, the methods include contacting a biological sample, e.g., a blood or cheek cell sample, with a compound or an agent capable of detecting p21 protein or a p21 nucleic acid, such that the presence of p21 nucleic acid or protein is detected in the biological sample.

In one embodiment, the compound or agent is a nucleic acid probe capable of hybridizing to p21 mRNA, or an antibody capable of binding to p21 protein.

In some embodiments, the evaluation is used to choose a course of treatment.

In another aspect, the invention features a computer readable record encoded with (a) a subject identifier, e.g., a patient identifier, (b) one or more results from an evaluation of the subject, e.g., a diagnostic evaluation described herein, e.g., the level of expression, level or activity of p21 in the subject, and optionally (c) a value for or related to a disease state, e.g., a value correlated with disease status or risk with regard to CAD or renal disease. In one embodiment, the invention features a computer medium having a plurality of digitally encoded data records. Each data record can include, e.g., a value indicating the presence or absence of a p21 polymorphism as described herein, a value representing the level of expression, level or activity of p21 in a sample, and a descriptor of the sample. The descriptor of the sample can be an identifier of the sample, a subject from which the sample was derived (e.g., a patient), a diagnosis, or a treatment (e.g., a preferred treatment). In one embodiment, the data record further includes values representing the level of expression, level or activity of genes other than p21 (e.g., other genes associated with renal disease or CAD, or other genes on an array). The data record can be structured as a table, e.g., a table that is part of a database such as a relational database (e.g., a SQL database of the Oracle or Sybase database environments). The invention also includes methods of communicating information about a subject, e.g., by transmitting information, e.g., transmitting a computer readable record described herein, e.g., over a computer network.

In another aspect, the invention features methods of providing information, e.g., for making a decision with regard to the treatment of a subject having, or at risk for, a disorder described herein. The methods include (a) evaluating the expression, level or activity of p21; optionally (b) providing a value for the expression, level or activity of p21; optionally (c) comparing the provided value with a reference value, e.g., a control or non-disease state reference or a disease state reference; and optionally (d) based, e.g., on the relationship of the provided value to the reference value, supplying information, e.g., information for making a decision on or related to the treatment of the subject.

In one embodiment, the provided value relates to an activity described herein, e.g., to p21 activity described herein.

In one embodiment, the decision is whether to administer a preselected treatment.

In one embodiment, the decision is whether a party, e.g., an insurance company, HMO, or other entity, will pay for all or part of a preselected treatment.

Also featured herein are methods of evaluating samples. The methods include providing a sample, e.g., comprising nucleic acid from a subject, and determining a gene expression profile of the sample, wherein the profile includes a value representing the level of expression of p21. The methods can further include comparing the value or the profile (i.e., multiple values) to a reference value or reference profile. The gene expression profile of the sample can be obtained by methods known in the art (e.g., by providing a nucleic acid from the sample and contacting the nucleic acid to an array). The method can be used to diagnose renal disease or CAD in a subject wherein misexpression of p21, e.g., an decrease in expression of p21, is an indication that the subject has a decreased risk of renal disease or CAD. The method can be used to monitor a treatment for renal disease or CAD, in a subject. For example, the gene expression profile can be determined for a sample from a subject undergoing treatment. The profile can be compared to a reference profile or to a profile obtained from the subject prior to treatment or prior to onset of the disorder (see, e.g., Golub et al., Science 286:531, (1999)).

In another aspect, the invention features methods of treating a subject, e.g., a human, having or at risk of developing renal disease or coronary artery disease (CAD). The methods include: identifying a subject having, or at risk of developing, renal disease or CAD, and administering to the subject an agent that decreases p21 signaling (e.g., decreases p21 expression, levels or activity).

The agent can be administered, e.g., orally, intravenously, percutaneously, subcutaneously, or implanted at a chosen site, e.g., in a cardiovascular tissue of the subject. The agent may be modified, e.g., to increase circulatory half-life, increase cellular uptake, improve distribution to target tissues (e.g., cardiovascular tissue), decrease clearance and/or decrease immunogenicity, e.g., as described herein. In one embodiment, the agent is administered in combination with another agent, e.g., another treatment for renal disease or CAD.

In one embodiment, the method can include contacting a cell, e.g., a cultured myocyte, with the agent in an amount sufficient to decrease p21 expression, levels or activity, and thereafter implanting the cell or cell population in a subject. In another embodiment, the agent is a cell, e.g., a cultured myocyte, that is genetically engineered in vitro to express an agent that decreases p21, and is then administered to the subject. The cells can be autologous, allogeneic or xenogeneic, but are preferably autologous. The cells can be implanted directly or can be administered in a scaffold, matrix, or other implantable device to which the cells can attach (examples include carriers made of, e.g., collagen, fibronectin, elastin, cellulose acetate, cellulose nitrate, polysaccharide, fibrin, gelatin, and combinations thereof).

In another embodiment, the agent is an nucleic acid antisense to a p21 nucleic acid.

In one embodiment, the method includes administering the agent in combination with a second treatment, e.g., a second treatment for renal disease or CAD.

In some embodiments, the method includes evaluating the subject for one or more of: heart contractility, heart rate, or ventricular function. The evaluation can be performed before, during, and/or after the administration of the agent. For example, the evaluation can be performed at least 1 day, 2 days, 4, 7, 14, 21, 30 or more days before and/or after the administration.

In some embodiments, the administration of an agent that decreases p21 expression, levels or activity can be initiated when the subject begins to show signs of renal disease or CAD; when renal disease or CAD is diagnosed; at the time a treatment for renal disease or CAD is begun or begins to exert its effects; or generally, as needed to maintain health.

The period over which the agent is administered (or the period over which clinically effective levels are maintained in the subject) can be long term, e.g., for six months or more or a year or more, or short term, e.g., for less than a year, six months, one month, two weeks or less.

An agent that decreases p21 signaling to thereby treat renal disease or CAD can be, for example a p21 binding protein, e.g., a soluble binding protein that binds p21 and inhibits a p21 activity, or inhibits the ability of a p21 to interact with a binding partner; an antibody that specifically binds to the p21 protein, e.g., an antibody that disrupts the ability of p21 to bind to a binding partner; a mutated inactive p21 or fragment thereof that disrupts a p21 activity (e.g., a dominant negative p21 mutant); a p21 nucleic acid molecule that can bind to a cellular p21 nucleic acid sequence, e.g., mRNA, and inhibit expression of the protein, e.g., an antisense, siRNA molecule or p21 ribozyme; and/or an agent that decreases p21 gene expression, e.g., a small molecule which binds and inhibits the promoter of p21. In one embodiment, p21 is inhibited by decreasing the level of expression of an endogenous p21 gene, e.g., by decreasing transcription of the p21 gene. In one embodiment, transcription of the p21 gene can be decreased by: altering the regulatory sequences of the endogenous p21 gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, the antibody that binds the p21 is a monoclonal antibody, e.g., a humanized chimeric or human monoclonal antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a list of p21 polymorphisms associated with decreased risk of renal disease. G−2266A is a G to A variance at position −2266 from the p21 transcription start site. G−1021A is a G to A variance at position −1021 from the p21 transcription start site. G+1004A is a G to A variance at position +1004 from the p21 transcription start site. The SNPs are shown in the context of approximately 200 nucleotides of surrounding genomic sequence.

FIG. 2 is a schematic illustrating part of the human p21 gene on chromosome 6. The underlined regions represent SEQ ID NOs:1, 2, and 3 as shown in FIG. 1; the SNPs are shown in bold face.

### DETAILED DESCRIPTION

Polymorphisms have been found in the p21 gene, e.g., in the p21 promoter, that are correlated with decreased risk of renal disease, e.g., a renal disease described herein, such as nephropathy or end stage renal disease (ESRD); with decreased risk of coronary artery disease (CAD); and with increased age of mortality.

Methods of Detecting p21 Polymorphisms

The methods described herein, e.g., diagnostic and prognostic methods described herein, can include evaluating one or more p21 polymorphisms.

Methods described herein provide for determining whether a subject carries a polymorphism of the p21 gene. For example, methods are provided for determining which allele or alleles of the human p21 gene a subject carries. Polymorphisms can be detected in a target nucleic acid from an individual. Samples that include p21 or the p21 gene can be utilized, e.g., blood samples. Genomic DNA, cDNA, mRNA, and/or proteins can be used to determine which of a plurality of polymorphisms are present in a subject.

Amplification of DNA from target samples can be accomplished by methods known to those of skill in the art, e.g., polymerase chain reaction (PCR). See, e.g., U.S. Pat. No. 4,683,202, ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA, 87:1874 (1990)) and nucleic acid based sequence amplification (NASBA). A variety of suitable procedures that can be employed to detect polymorphisms are described in further detail below.

Allele-Specific Probes

The design and use of allele-specific probes for analyzing polymorphisms is known in the art (see, e.g., Dattagupta, EP 235,726, Saiki, WO 89/11548). Allele-specific probes can be designed to hybridize differentially, e.g., to hybridize to a segment of DNA from one individual but not to a corresponding segment from another individual, based on the presence of polymorphic forms of the segment. Relatively stringent hybridization conditions can be utilized to cause a significant difference in hybridization intensity between alleles, and possibly to obtain a condition wherein a probe hybridizes to only one of the alleles. Probes can be designed to hybridize to a segment of DNA such that the polymorphic site aligns with a central position of the probe.

Allele-specific probes can be used in pairs, wherein one member of the pair matches perfectly to a reference form of a target sequence, and the other member of the pair matches perfectly to a variant of the target sequence. The use of several pairs of probes immobilized on the same support may allow simultaneous analysis of multiple polymorphisms within the same target sequence.

Tiling Arrays

Polymorphisms can also be identified by hybridization to nucleic acid arrays (see, e.g., WO 95/11995). WO 95/11995 also describes subarrays that are optimized for detection of variant forms of a pre-characterized polymorphism. Such a subarray contains probes designed to be complementary to a second reference sequence, which is an allelic variant of the first reference sequence. The second group of probes is designed to exhibit complementarily to the second reference sequence. The inclusion of a second group (or further groups) can be particularly useful for analyzing short subsequences of the primary reference sequence in which multiple mutations are expected to occur within a short distance commensurate with the length of the probes (i.e., two or more mutations within 9 to 21 bases).

Allele-Specific Primers

An allele-specific primer hybridizes to a site on target DNA overlapping a polymorphism and only primes amplification of an allelic form to which the primer exhibits perfect complementarily. See, e.g., Gibbs, Nucleic Acid Res. 17:2427-2448 (1989). Such a primer can be used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method can be optimized by including the mismatch in the 3′-most position of the oligonucleotide aligned with the polymorphism because this position is most destabilizing to elongation from the primer. See, e.g., WO 93/22456.

Direct-Sequencing

The direct analysis of the sequence of polymorphisms of the present invention can be accomplished using either the dideoxy chain termination method or the Maxam Gilbert method (see Sambrook et al., Molecular Cloning: A Laboratory Manual, 3d ed., Cold Spring Harbor, 2001; Zyskind et al., Recombinant DNA Laboratory Manual, (Acad. Press, 1988)).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86:2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

Other methods of detecting polymorphisms, e.g., SNPs, are known, e.g., as described in U.S. Pat. No. 6,410,231; U.S. Pa. No. 6,361,947; U.S. Pat. No. 6,322,980; U.S. Pat. No. 6,316,196; U.S. Pat. No. 6,258,539.

Detection Of Variations Or Mutations

Alterations or mutations in a p21 gene can be identified by a number of methods known in the art, to thereby identify other polymorphisms that may be associated with susceptibility for renal disease or CAD. In some embodiments, the methods include detecting, in a sample from the subject, the presence or absence of a genetic alteration characterized by an alteration affecting the integrity of a gene encoding a p21 protein, or the mis-expression of the p21 gene. For example, such genetic alterations can be detected by ascertaining the existence of at least one of 1) a deletion of one or more nucleotides from a p21 gene; 2) an addition of one or more nucleotides to a p21 gene; 3) a substitution of one or more nucleotides of a p21 gene, 4) a chromosomal rearrangement of a p21 gene; 5) an alteration in the level of a messenger RNA transcript of a p21 gene, 6) aberrant modification of a p21 gene, such as of the methylation pattern of the genomic DNA, 7) the presence of a non-wild type splicing pattern of a messenger RNA transcript of a p21 gene, 8) a non-wild type level of a p21-protein, 9) allelic loss of a p21 gene, and 10) inappropriate post-translational modification of a p21-protein.

An alteration can be detected with or without a probe/primer in a polymerase chain reaction, e.g., by anchor PCR or RACE PCR, or, alternatively, in a ligation chain reaction (LCR), the latter of which can be particularly useful for detecting point mutations in the p21 gene. This method can include the steps of collecting a sample of cells from a subject, isolating nucleic acid (e.g., genomic, MRNA or both) from the sample, contacting the nucleic acid sample with one or more primers which specifically hybridize to a p21 gene under conditions such that hybridization and amplification of the p21 gene (if present) occurs, and detecting the presence or absence of an amplification product, or detecting the size of the amplification product and comparing the length to a control sample. PCR and/or LCR can be used as a preliminary amplification step in conjunction with any of the techniques used for detecting mutations described herein. Alternatively, other amplification methods described herein or known in the art can be used.

In another embodiment, mutations in a p21 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in p21 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a p21 nucleic acid or a putative variant (e.g., allelic variant) thereof. A probe can have one or more mismatches to a region of a p21 nucleic acid (e.g., a destabilizing mismatch). The arrays can have a high density of addresses, e.g., can contain hundreds or thousands of oligonucleotides probes (Cronin, M. T. et al., Human Mutation 7:244-255(1996); Kozal et al., Nature Medicine 2: 753-759, (1996)). For example, genetic mutations in p21 can be identified in two-dimensional arrays containing light-generated DNA probes as described in Cronin, M. T. et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the p21 gene and detect mutations by comparing the sequence of the sample p21 with the corresponding wild-type (control) sequence. Automated sequencing procedures can be utilized when performing the diagnostic assays (Naeve et al., Biotechniques 19:448-453 (1995)), including sequencing by mass spectrometry.

Other methods for detecting mutations in the p21 gene include methods in which protection from cleavage agents is used to detect mismatched bases in RNA/RNA or RNA/DNA heteroduplexes (Myers et al., Science 230:1242 (1985); Cotton et al., Proc. Natl Acad Sci USA 85:4397 (1988); Saleeba et al., Methods Enzymol. 217:286-295 (1992)).

In still another embodiment, the mismatch cleavage reaction employs one or more proteins that recognize mismatched base pairs in double-stranded DNA (so called “DNA mismatch repair” enzymes) in defined systems for detecting and mapping point mutations in p21 cDNAs obtained from samples of cells. For example, the mutY enzyme of E. coli cleaves A at G/A mismatches and the thymidine DNA glycosylase from HeLa cells cleaves T at G/T mismatches (Hsu et al., Carcinogenesis 15:1657-1662 (1994); U.S. Pat. No. 5,459,039).

In other embodiments, alterations in electrophoretic mobility will be used to identify mutations in p21 genes. For example, single strand conformation polymorphism (SSCP) may be used to detect differences in electrophoretic mobility between mutant and wild type nucleic acids (Orita et al., Proc Natl. Acad. Sci USA: 86:2766 (1989), see also Cotton Mutat. Res. 285:125-144 (1993); and Hayashi Genet. Anal. Tech. Appl. 9:73-79 (1992)). Single-stranded DNA fragments of sample and control p21 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., Trends Genet 7:5 (1991)).

In yet another embodiment, the movement of mutant or wild-type fragments in polyacrylamide gels containing a gradient of denaturant is assayed using denaturing gradient gel electrophoresis (DGGE) (Myers et al., Nature 313:495 (1985)). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner Biophys Chem 265:12753 (1987)).

Examples of other techniques for detecting point mutations include, but are not limited to, selective oligonucleotide hybridization, selective amplification, or selective primer extension (Saiki et al., Nature 324:163-66 (1986)); Saiki et al., Proc. Natl Acad. Sci USA 86:6230-34 (1989)). A further method of detecting point mutations is the chemical ligation of oligonucleotides as described in Xu et al., Nature Biotechnol. 19:148-152 (2001). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al., Nucleic Acids Res. 17:2437-2448 (1989)) or at the extreme 3′ end of one primer where, under appropriate conditions, mismatch can prevent, or reduce polymerase extension (Prossner, Tibtech 11:238 (1993)). In addition it may be desirable to introduce a novel restriction site in the region of the mutation to create cleavage-based detection (Gasparini et al., Mol. Cell Probes 6(1):1-7 (1992)). It is anticipated that in certain embodiments amplification may also be performed using Taq ligase for amplification (Barany, Proc. Natl. Acad. Sci USA 88:189-93 (1991)). In such cases, ligation will occur only if there is a perfect match at the 3′ end of the 5′ sequence making it possible to detect the presence of a known mutation at a specific site by looking for the presence or absence of amplification.

Antisense Nucleic Acid Sequences

A nucleic acid molecule that is antisense to a a p21 nucleic acid, can be used as an agent to inhibit expression of p21, e.g., in a subject having or at risk for CAD or a renal disease, e.g., a renal disease described herein. An “antisense” nucleic acid includes a nucleotide sequence which is complementary to a “sense” nucleic acid encoding the component, e.g., complementary to the coding strand of a double-stranded cDNA molecule or complementary to an MRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule which antisense to the “coding region” of the coding strand of a nucleotide sequence encoding the component can be used.

The sequence of the p21 gene is known (Genbank Accession No. Z85996.1). Given the gene sequences, antisense nucleic acids can be designed according to the rules of Watson and Crick base pairing. An antisense nucleic acid molecule can be, e.g., complementary to all or part of the coding region of mRNA, but more preferably is antisense to only a portion of the coding or noncoding region of mRNA. For example, the antisense oligonucleotide can be complementary to the region surrounding the translation start site of the MRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides that can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′ -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically, e.g., using an expression vector into which a nucleic acid has been subcloned in an antisense orientation. RNA transcribed from the inserted nucleic acid will be of an antisense orientation to a target nucleic acid of interest.

RNAi

Double stranded nucleic acid molecules that can silence a gene encoding p21 can also be used as an agent to inhibit expression of the p21. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect can persist for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al., Nature 411(6836):494-8 (2001)). In one embodiment, gene silencing can be induced in mammalian cells by enforcing endogenous expression of RNA hairpins (see Paddison et al., Proc. Nat. Acad. Sci. USA 99:1443-1448 (2002)). In another embodiment, transfection of small (21-23 nt) dsRNA specifically inhibits gene expression (reviewed in Caplen, Trends in Biotechnology 20:49-51 (2002)).

Briefly, RNAi is thought to work as follows. dsRNA corresponding to a portion of a gene to be silenced is introduced into a cell. The dsRNA is digested into 21-23 nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA ˜12 nucleotides from the 3′ terminus of the siRNA (reviewed in Sharp et al Genes Dev 15: 485-490 (2001); and Hammond et al., Nature Rev Gen 2:110-119 (2001)).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem. Biophys. Res. Commun. 281(3):639-44 (2001)), providing yet another strategy for gene silencing.

Therapeutic applications of RNAi are described, e.g., in Shuey, Drug Discov Today 7(20):1040-6 (2002).

Antibodies

In another aspect, an anti-p21 antibody, e.g., an inhibitory antibody, can be used to decrease p21 expression, levels or activity in the methods described herein.

The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat et al., Sequences of Proteins of Immunological Interest, Fifth Edition, U.S. Department of Health and Human Services, NIH Publication No. 91-3242 (1991), and Chothia, C. et al., J. Mol. Biol. 196:901-917 (1987)). Each VH and VL is composed of three CDRs and four FRs, arranged from amino-terminus to carboxy-terminus in the following order: FR1, CDR1, FR2, CDR2, FR3, CDR3, FR4.

The anti-p21 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “antigen-binding fragment” of an antibody (or simply “antibody portion,” or “fragment”), as used herein, refers to one or more fragments of a full-length antibody that retain the ability to specifically bind to the antigen, e.g., p21 polypeptide or fragment thereof. Examples of antigen-binding fragments of the anti-p21 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science 242:423-426 (1988); and Huston et al., Proc. Natl. Acad. Sci. USA 85:5879-5883 (1988)). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-p21 antibody can be a polyclonal or a monoclonal antibody. In other embodiments, the antibody can be recombinantly produced, e.g., produced by phage display or by combinatorial methods.

Phage display and combinatorial methods for generating anti-p21 antibodies are known in the art (as described in, e.g., Ladner et al., U.S. Pat. No. 5,223,409; Kang et al., International Publication No. WO 92/18619; Dower et al., International Publication No. WO 91/17271; Winter et al., International Publication WO 92/20791; Markland et al., International Publication No. WO 92/15679; Breitling et al., International Publication WO 93/01288; McCafferty et al., International Publication No. WO 92/01047; Garrard et al., International Publication No. WO 92/09690; Ladner et al., International Publication No. WO 90/02809; Fuchs et al., Bio/Technology 9:1370-1372 (1991); Hay et al., Hum Antibod Hybridomas 3:81-85 (1992); Huse et al., Science 246:1275-1281 (1989); Griffths et al., EMBO J 12:725-734 (1993); Hawkins et al., J Mol Biol 226:889-896 (1992); Clackson et al., Nature 352:624-628 (1991); Gram et al., Proc. Nat. Acad. Sci. USA 89:3576-3580 (1992); Garrad et al., Bio/Technology 9:1373-1377 (1991); Hoogenboom et al., Nuc Acid Res 19:4133-4137 (1991); and Barbas et al., Proc. Nat. Acad. Sci. USA 88:7978-7982 (1991)).

In one embodiment, the anti-p21 antibody is a fully human antibody (e.g., an antibody made in a mouse that has been genetically engineered to produce an antibody with a human immunoglobulin sequence), or a non-human antibody, e.g., a rodent (mouse or rat), goat, primate (e.g., monkey), camel antibody. Typically, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al., International Application WO 91/00906, Kucherlapati et al., PCT publication WO 91/10741; Lonberg et al., International Application WO 92/03918; Kay et al., International Application 92/03917; Lonberg et al., Nature 368:856-859 (1994); Green et al., Nature Genet. 7:13-21 (1994); Morrison et al., Proc. Natl. Acad. Sci. USA 81:6851-6855 (1994); Bruggeman et al., Year Immunol 7:33-40 (1993); Tuaillon et al., Proc. Natl. Acad. Sci. 90:3720-3724 (1993); Bruggeman et al., Eur J Immunol 21:1323-1326 (1991)).

An anti-p21 antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira et al., European Patent Application 184,187; Taniguchi, European Patent Application 171,496; Morrison et al., European Patent Application 173,494; Neuberger et al., International Application WO 86/01533; Cabilly et al., U.S. Pat. No. 4,816,567; Cabilly et al., European Patent Application 125,023; Better et al., Science 240:1041-1043 (1988); Liu et al., Proc. Nat. Acad. Sci. USA 84:3439-3443 (1987); Liu et al., J. Immunol. 139:3521-3526 (1987); Sun et al., Proc. Nat. Acad. Sci. USA 84:214-218 (1987); Nishimura et al., Canc. Res. 47:999-1005 (1987); Wood et al., Nature 314:446-449 (1985); and Shaw et al., J. Natl Cancer Inst. 80:1553-1559 (1988)).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to a p21 or a fragment thereof. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence at least about 85%, e.g., 90%, 95%, 99% or more, identical thereto.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., BioTechniques 4:214 (1986), and by Queen et al., U.S. Pat. No. 5,585,089, U.S. Pat. No. 5,693,761 and U.S. Pat. No. 5,693,762. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a p21 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al., Nature 321:552-525 (1986); Verhoeyan et al., Science 239:1534 (1988); Beidler et al., J. Immunol. 141:4053-4060 (1988); Winter U.S. Pat. No. 5,225,539. Winter describes a CDR-grafting method which may be used to prepare the humanized antibodies of the present invention (UK Patent GB 2188638A; Winter, U.S. Pat. No. 5,225,539).

A full-length p21 protein or antigenic peptide fragment of p21 can be used as an immunogen or can be used to identify anti-p21 antibodies made with other immunogens, e.g., cells, membrane preparations, and the like. The antigenic peptide of p21 should include at least 8 amino acid residues of the p21 amino acid sequence, e.g., the human p21 amino acid sequence and encompasses an epitope of p21. Typically, the antigenic peptide will include at least 10 amino 15, 20 or 30 amino acid residues.

Antibodies reactive with, or specific for, any of these regions, or other regions or domains described herein are provided.

Antibodies that bind only native p21 protein, only denatured or otherwise non-native p21 protein, or that bind both, can be used in the methods described herein. Antibodies with linear or conformational epitopes can also be used. Conformational epitopes can sometimes be identified by identifying antibodies that bind to native but not denatured p21 protein.

Preferred epitopes encompassed by the antigenic peptide are regions of p21 located on the surface of the protein, e.g., hydrophilic regions, as well as regions with high antigenicity. For example, an Emini surface probability analysis of the human p21 protein sequence can be used to indicate the regions that have a particularly high probability of being localized to the surface of the p21 protein and are thus likely to constitute surface residues useful for targeting antibody production.

The anti-p21 antibody can be a single chain antibody. A single-chain antibody (scFV) may be engineered (see, for example, Colcher et al., Ann. N.Y. Acad. Sci. 880:263-80 (1999); and Reiter Clin Cancer Res 2:245-52 (1996)). The single chain antibody can be dimerized or multimerized to generate multivalent antibodies having specificities for different epitopes of the same target p21 protein.

In one embodiment, the antibody has effector function and/or can fix complement. In other embodiments the antibody does not recruit effector cells or fix complement.

In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

In some embodiments, an anti-p21 antibody alters (e.g., decreases) a p21 activity described herein.

The antibody can be coupled to a toxin, e.g., a polypeptide toxin, e,g, ricin or diphtheria toxin or active fragment hereof, or a radioactive nucleus, or imaging agent, e.g. a radioactive, enzymatic, or other, e.g., imaging agent, e.g., a NMR contrast agent. Labels which produce detectable radioactive emissions or fluorescence are preferred.

An anti-p21 antibody (e.g., monoclonal antibody) can be used to isolate p21 by standard techniques, such as affinity chromatography or immunoprecipitation. Moreover, an anti-p21 antibody can be used to detect p21 protein (e.g., in a cellular lysate or cell supernatant) in order to evaluate the abundance and pattern of expression of the protein. Anti-p21 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Administration

An agent that modulates p21 signaling, e.g., an agent described herein, such as a p21 antibody or antisense molecule, can be administered to a subject by standard methods. For example, the agent can be administered by any of a number of different routes including intravenous, intradermal, subcutaneous, percutaneous injection, oral (e.g., inhalation), transdermal (topical), and transmucosal. In one embodiment, the modulating agent can be administered orally. In another embodiment, the agent is administered by injection, e.g., intramuscularly, or intravenously. The agent may be encapsulated or injected, e.g., in a viscous form, for delivery to a chosen site. The agent can be provided in a matrix capable of delivering the agent to the chosen site. Matrices can provide slow release of the agent and provide proper presentation and appropriate environment for cellular infiltration. Matrices may be formed of materials presently in use for other implanted medical applications. The choice of matrix material is based on any one or more of: biocompatibility, biodegradability, mechanical properties, cosmetic appearance and interface properties. One example is a collagen matrix.

The agent, e.g., p21 polypeptide, nucleic acid molecule, analog, mimetic or modulators (e.g., organic compounds or antibodies (also referred to herein as “active compounds”) can be incorporated into pharmaceutical compositions suitable for administration to a subject, e.g., a human. Such compositions typically include the polypeptide, nucleic acid molecule, modulator, or antibody and a pharmaceutically acceptable carrier. As used herein the language “pharmaceutically acceptable carrier,” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., Proc. Nat. Acad. Sci. USA 91:3054-3057 (1994)). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

In a preferred embodiment, the pharmaceutical composition is injected into a tissue, e.g., a kidney tissue.

Gene Therapy

The nucleic acids described herein, e.g., a p21 antisense nucleic acid described herein, or p21 polypeptide-encoding nucleic acid, can be incorporated into a gene construct to be used as a part of a gene therapy protocol to deliver nucleic acids encoding either an agonistic or antagonistic form of an agent described herein, e.g., p21. The invention features expression vectors for in vivo transfection and expression of a p21 polypeptide described herein in particular cell types. Such expression constructs can be administered in any biologically effective carrier, e.g., any formulation or composition capable of effectively delivering the component gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation carried out in vivo.

One approach for in vivo introduction of nucleic acid into a cell is by use of a viral vector containing nucleic acid, e.g. a cDNA, encoding an alternative pathway component described herein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) that produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel et al. (eds.), Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al., Science 230:1395-1398 (1985); Danos and Mulligan Proc. Natl. Acad. Sci. USA 85:6460-6464 (1988); Wilson et al., Proc. Natl. Acad. Sci. USA 85:3014-3018 (1988); Armentano et al., Proc. Natl. Acad. Sci. USA 87:6141-6145 (1990); Huber et al., Proc. Natl. Acad. Sci. USA 88:8039-8043 (1991); Ferry et al., Proc. Natl. Acad. Sci. USA 88:8377-8381 (1991); Chowdhury et al., Science 254:1802-1805 (1991); van Beusechem et al., Proc. Natl. Acad. Sci. USA 89:7640-7644 (1992); Kay et al., Human Gene Therapy 3:641-647 (1992); Dai et al., Proc. Natl. Acad. Sci. USA 89:10892-10895 (1992); Hwu et al., J. Immunol. 150:4104-4115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., cited supra; Haj-Ahmand and Graham, J. Virol. 57:267 (1986)).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol.158:97-129 (1992)). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993)).

In addition to viral transfer methods, such as those illustrated above, non-viral methods can also be employed to cause expression of an nucleic acid agent described herein (e.g., a p21 polypeptide encoding nucleic acid) in the tissue of a subject. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000 ).

In a representative embodiment, a gene encoding an alternative pathway component described herein can be entrapped in liposomes bearing positive charges on their surface (e.g., lipofectins) and (optionally) which are tagged with antibodies against cell surface antigens of the target tissue (Mizuno et al., No Shinkei Geka 20:547-551 (1992); PCT publication W091/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., Proc. Nat. Acad. Sci. USA 91: 3054-3057 (1994)).

A pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Diagnostic Assays

The diagnostic assays described herein involve evaluating p21 expression, levels, or activity in the subject, e.g., in a kidney or cardiovascular tissue. Various art-recognized methods are available for evaluating the activity of p21 or the p21 signaling pathway or components thereof. For example, the method can include evaluating either the level of a p21 pathway component (e.g., the level of p21) and/or an activity of the p21 pathway. Techniques for detection of p21 are known in the art and include, inter alia,: antibody-based assays such as enzyme immunoassays (EIA), radioimmunoassays (RIA), and Western blot analysis. Typically, the level in the subject is compared to the level and/or activity in a control, e.g., the level and/or activity in a tissue from a non-disease subject.

Techniques for evaluating binding activity, e.g., of p21 to a p21 binding partner include fluid phase binding assays, affinity chromatography, size exclusion or gel filtration, ELISA, immunoprecipitation (e.g., the ability of an antibody specific to a first factor, e.g., p21, to co-immunoprecipitate a second factor or complex, with which the first factor can associate in nature).

Another method of evaluating the p21 pathway in a subject is to determine the presence or absence of a lesion in, or the misexpression of, a gene that encodes a component of the p21 pathway, e.g., p21. The method includes one or more of the following:
detecting, in a tissue of the subject, the presence or absence of a mutation which affects the expression of a gene encoding p21, or detecting the presence or absence of a mutation in a region that controls the expression of the gene, e.g., a mutation in the 5′ control region;
detecting, in a tissue of the subject, the presence or absence of a mutation that alters the structure of a gene encoding p21;
detecting, in a tissue of the subject, the misexpression of a gene encoding p21, at the mRNA level, e.g., detecting a non-wild type level of a mRNA;
detecting, in a tissue of the subject, the misexpression of the gene, at the protein level, e.g., detecting a non-wild type level of a p21 polypeptide.

In preferred embodiments the method includes: ascertaining the existence of at least one of a deletion of one or more nucleotides from a gene encoding p21; an insertion of one or more nucleotides into the gene; a point mutation, e.g., a substitution of one or more nucleotides of the gene as described herein; and/or a gross chromosomal rearrangement of the gene, e.g., a translocation, inversion, or deletion.

For example, detecting the genetic lesion can include: (i) providing a probe/primer including an oligonucleotide containing a region of nucleotide sequence that hybridizes to a sense or antisense sequence from a p21 gene, or naturally occurring mutants thereof, or 5′ or 3′ flanking sequences naturally associated with the gene, e.g., as described herein; (ii) exposing the probe/primer to nucleic acid of a tissue; and detecting hybridization, e.g., in situ hybridization, of the probe/primer to the nucleic acid, the presence or absence of the genetic lesion.

In some embodiments detecting the misexpression includes ascertaining the existence of at least one of an alteration in the level of a messenger RNA transcript of a gene encoding p21; the presence of a non-wild type splicing pattern of a messenger RNA transcript of the gene; and/or a non-wild type level of a gene encoding p21.

In some embodiments the method includes determining the structure of a gene encoding p21, an abnormal structure being indicative of risk for the disorder.

In some embodiments the method includes contacting a sample from the subject with an antibody to p21, or a nucleic acid that hybridizes specifically with the p21 gene or a polymorphic variant thereof, e.g., as described herein.

Expression Monitoring and Profiling.

The activity, presence, level, or absence of p21 (protein or nucleic acid) in a biological sample can be evaluated by obtaining a biological sample from a test subject and contacting the biological sample with a compound or an agent capable of detecting the protein or nucleic acid (e.g., mRNA, genomic DNA) that encodes p21, such that the presence of the protein or nucleic acid is detected in the biological sample. The term “biological sample” includes tissues, cells and biological fluids isolated from a subject, as well as tissues, cells and fluids present within a subject, e.g., blood. Preferred biological samples are serum or epithelial, e.g., cheek cells. The level of expression of p21 can be measured in a number of ways, including, but not limited to measuring the mRNA encoded by the p21 gene; measuring the amount of protein encoded by p21; and/or measuring the activity of the protein encoded by the gene.

The level of mRNA corresponding to p21 in a cell can be determined both by in situ and by in vitro formats.

Isolated mRNA or genomic DNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length nucleic acid, or a portion thereof, such as an oligonucleotide of at least 7, 15, 30, 50, 100, 250 or 500 nucleotides in length and sufficient to specifically hybridize under stringent conditions to mRNA or genomic DNA of p21. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated MRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of MRNA encoded by the gene os a component of the alternative pathway.

The level of mRNA in a sample that is encoded by a gene can be evaluated with nucleic acid amplification, e.g., by rtPCR (Mullis, U.S. Pat. No. 4,683,202), ligase chain reaction (Barany, Proc. Natl. Acad. Sci. USA 88:189-193 (1991)), self sustained sequence replication (Guatelli et al., Proc. Natl. Acad. Sci. USA 87:1874-1878 (1990)), transcriptional amplification system (Kwoh et al., Proc. Natl. Acad. Sci. USA 86:1173-1177 (1989)), Q-Beta Replicase (Lizardi et al., Bio/Technology 6:1197 (1988)), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to MRNA that encodes the gene being analyzed.

In another embodiment, the methods further contacting a control sample with a compound or agent capable of detecting MRNA, or genomic DNA of p21, and comparing the presence of the mRNA or genomic DNA in the control sample with the presence of p21 mRNA or genomic DNA in the test sample. In still another embodiment, serial analysis of gene expression, as described in U.S. Pat. No. 5,695,937, can be used to detect transcript levels of p21.

A variety of methods can be used to determine the level of p21 protein. In general, these methods include contacting a sample with an agent that selectively binds to the protein, such as an antibody, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods described herein can be used to detect p21 or polymorphic variants thereof in a biological sample in vitro as well as in vivo. In vitro techniques for detection include enzyme linked immunosorbent assays (ELISAs), immunoprecipitations, immunofluorescence, enzyme immunoassay (EIA), radioimmunoassay (RIA), and Western blot analysis. In vivo techniques for detection include introducing into a subject a labeled antibody. For example, the antibody can be labeled with a radioactive marker whose presence and location in a subject can be detected by standard imaging techniques. In another embodiment, the sample is labeled, e.g., biotinylated and then contacted to the antibody, e.g., an antibody positioned on an antibody array. The sample can be detected, e.g., with avidin coupled to a fluorescent label.

In another embodiment, the methods further include contacting the control sample with a compound or agent capable of detecting a p21, and comparing the presence of p21 protein in the control sample with the presence of the protein in the test sample.

The invention also includes kits for detecting the presence of p21 or a polymorphic variant thereof in a biological sample. For example, the kit can include a compound or agent capable of detecting p21 protein (e.g., an antibody) genomic DNA or mRNA (e.g., a nucleic acid probe); and a standard. The compound or agent can be packaged in a suitable container. The kit can further comprise instructions for using the kit to evaluate a subject, e.g., for risk or predisposition to renal or cardiovascular disease, e.g., CAD.

The diagnostic methods described herein can identify subjects having, or at risk of developing, renal related disorders, such as nephropathy, ESRD or CAD. The prognostic assays described herein can be used to determine whether a subject should be administered an agent (e.g., a p21 inhibiting agent described herein) to treat a renal-related or CAD disorder.

Kits

A p21 nucleic acid or polypeptide described herein, or an agent that modulates p21, such as a p21 inhibitory antibody, can be provided in a kit. The kit includes (a) the agent, e.g., p21 or p21 antibody, and (b) informational material. The informational material can be descriptive, instructional, marketing or other material that relates to the methods described herein and/or the use of p21 for the methods described herein. For example, the informational material relates to renal disease or CAD.

In one embodiment, the informational material can include instructions to administer the inhibiting polypeptide or nucleic acid p21 in a suitable manner to perform the methods described herein, e.g., in a suitable dose, dosage form, or mode of administration (e.g., a dose, dosage form, or mode of administration described herein). In another embodiment, the informational material can include instructions to administer the p21 or p21 inhibiting agent to a suitable subject, e.g., a human, e.g., a human having, or at risk for, renal disease or CAD.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about p21 and/or its use in the methods described herein. Of course, the informational material can also be provided in any combination of formats.

In addition to p21 or agents that modulate p21, the compositions of the kits can include other ingredients, such as a solvent or buffer, a stabilizer, a preservative, a fragrance or other cosmetic ingredient, and/or a second agent for treating a condition or disorder described herein, e.g., renal disease or CAD. Alternatively, the other ingredients can be included in the kit, but in different compositions or containers than the agent. In such embodiments, the kit can include instructions for admixing the agent and the other ingredients, or for using the agent together with the other ingredients.

The agent can be provided in any form, e.g., liquid, dried or lyophilized form. It is preferred that the agent be substantially pure and/or sterile. When the agent is provided in a liquid solution, the liquid solution preferably is an aqueous solution, with a sterile aqueous solution being preferred. When the agent is provided as a dried form, reconstitution generally is by the addition of a suitable solvent. The solvent, e.g., sterile water or buffer, can optionally be provided in the kit.

The kit can include one or more containers for the composition containing the agent. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agent. For example, the kit includes a plurality of syringes, ampules, foil packets, or blister packs, each containing a single unit dose of the agent. The containers of the kits can be air tight and/or waterproof.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device.

Generation of Variants: Production of Altered DNA and Peptide Sequences by Random Methods

Amino acid sequence variants of p21 polypeptides or fragments thereof can be prepared by a number of techniques, such as random mutagenesis of DNA which encodes a p21 or a region thereof. Useful methods also include PCR mutagenesis and saturation mutagenesis. A library of random amino acid sequence variants can also be generated by the synthesis of a set of degenerate oligonucleotide sequences.

PCR Mutagenesis

In PCR mutagenesis, reduced Taq polymerase fidelity is used to introduce random mutations into a cloned fragment of DNA (Leung et al., Technique 1:11-15 (1989)). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Mayers et al., Science 229:242 (1985)). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, Tetrahedron 39:3 (1983); Itakura et al., Recombinant DNA, Proc 3rd Cleveland Sympos. Macromolecules, Walton, Ed., Amsterdam: Elsevier (1981) pp. 273-289; Itakura et al., Annu. Rev. Biochem. 53:323 (1984); Itakura et al., Science 198:1056 (1984); Ike et al., Nucleic Acid Res. 11:477 (1983). Such techniques have been employed in the directed evolution of other proteins (see, for example, Scott et al., Science 249:386-390 (1990); Roberts et al., Proc. Nat. Acad. Sci. USA 89:2429-2433 (1992); Devlin et al., Science 249: 404-406 (1990); Cwirla et al., Proc. Nat. Acad. Sci. USA 87:6378-6382 (1990); as well as U.S. Pat. Nos. 5,223,409, 5,198,346, and 5,096,815).

Generation of Variants: Production of Altered DNA and Peptide Sequences by Directed Mutagenesis

Non-random or directed mutagenesis techniques can be used to provide specific sequences or mutations in specific regions. These techniques can be used to create variants that include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085 (1989)). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA, see, e.g., Adelman et al., (DNA 2:183, (1983)). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., (Proc. Natl. Acad. Sci. USA, 75: 5765 (1978)).

Cassette Mutagenesis

Another method for preparing variants, cassette mutagenesis, is based on the technique described by Wells et al., (Gene, 34:315 (1985)). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate variants. For example, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High Through Put Methods for Screening Libraries of Peptide Fragments or Homologs

Various techniques are known in the art for screening peptides, e.g., synthetic peptides, e.g., small molecular weight peptides (e.g., linear or cyclic peptides) or generated mutant gene products. Techniques for screening large gene libraries often include cloning the gene library into replicable expression vectors, transforming appropriate cells with the resulting library of vectors, and expressing the genes under conditions in which detection of a desired activity, assembly into a trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid (interaction trap) assays can be used to identify a protein that interacts with p21. These may include, e.g., agonists, superagonists, and antagonists of p21. (The subject protein and a protein it interacts with are used as the bait protein and fish proteins.). These assays rely on detecting the reconstitution of a functional transcriptional activator mediated by protein-protein interactions with a bait protein. In particular, these assays make use of chimeric genes which express hybrid proteins. The first hybrid comprises a DNA-binding domain fused to the bait protein. e.g., p21 or active fragments thereof. The second hybrid protein contains a transcriptional activation domain fused to a “fish” protein, e.g. an expression library. If the fish and bait proteins are able to interact, they bring into close proximity the DNA-binding and transcriptional activator domains. This proximity is sufficient to cause transcription of a reporter gene which is operably linked to a transcriptional regulatory site which is recognized by the DNA binding domain, and expression of the marker gene can be detected and used to score for the interaction of the bait protein with another protein.

Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al., Bio/Technology 9:1370-1371 (1991); and Goward et al., TIBS 18:136-140 (1992)). This technique was used in Sahu et al., J. Immunology 157:884-891 (1996), to isolate a complement inhibitor. In a similar fashion, a detectably labeled ligand can be used to score for potentially functional peptide homologs. Fluorescently labeled ligands, e.g., receptors, can be used to detect homolog which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and f1 are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al., PCT publication WO 90/02909; Garrard et al., PCT publication WO 92/09690; Marks et al., J. Biol. Chem. 267:16007-16010 (1992); Griffiths et al., EMBO J 12:725-734 (1993); Clackson et al., Nature 352:624-628 (1991); and Barbas et al., Proc. Nat. Acad. Sci. USA 89:4457-4461 (1992)).

A common approach uses the maltose receptor of E. coli (the outer membrane protein, LamB) as a peptide fusion partner (Charbit et al., EMBO 5, 3029-3037 (1986)). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al., Vaccines 91:387-392 (1991)), PhoE (Agterberg, et al., Gene 88:37-45 (1990)), and PAL (Fuchs et al., Bio/Tech 9:1369-1372 (1991)), as well as large bacterial surface structures have served as vehicles for peptide display. Peptides can be fused to pilin, a protein which polymerizes to form the pilus-a conduit for interbacterial exchange of genetic information (Thiry et al., Appl. Environ. Microbiol. 55:984-993 (1989)). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al., Bio/Tech. 6:1080-1083 (1988)). Surface proteins of other bacterial species have also served as peptide fusion partners. Examples include the Staphylococcus protein A and the outer membrane protease IgA of Neisseria (Hansson et al., J. Bacteriol. 174:4239-4245 (1992) and Klauser et al., EMBO J. 9, 1991-1999 (1990)).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al., Proc. Nat. Acad. Sci. USA 89:1865-1869 (1992)). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al., Proc. Natl. Acad. Sci. U.S.A. 89-1869 (1992))

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pill and pVIII, are anchored to the phage through their C-termini, and the guest peptides are placed into the outward-extending N-terminal domains. In some designs, the phage-displayed peptides are presented right at the amino terminus of the fusion protein. (Cwirla, et al., Proc. Natl. Acad. Sci. U.S.A. 87:6378-6382 (1990)). A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al., J. Med. Chem. 37(9):1233-1251 (1994)). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al., J. Med. Chem. 37(9):1233-1251 (1994)), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al., Anal. Biochem 204:357-364 (1992)). To identify the sequences of the active peptides one sequences the DNA produced by the phagemid host.

Secondary Screens

The high through put assays described above can be followed (or substituted) by secondary screens in order to identify biological activities which will, e.g., allow one skilled in the art to differentiate agonists from antagonists. The type of a secondary screen used will depend on the desired activity that needs to be tested.

Peptide Mimetics

The invention also provides for production of the protein binding domains of p21, to generate mimetics, e.g. peptide or non-peptide agents, e.g., agonists or antagonists.

Non-hydrolyzable peptide analogs of critical residues can be generated using benzodiazepine (e.g., see Freidinger et al., in Peptides: Chemistry and Biology, Marshall, Ed., ESCOM Publisher: Leiden, Netherlands, 1988), azepine (e.g., see Huffinan et al., in Peptides: Chemistry and Biology, Marshall, Ed., ESCOM Publisher: Leiden, Netherlands, 1988), substituted gamma lactam rings (Garvey et al., in Peptides: Chemistry and Biology, Marshall, Ed., ESCOM Publisher: Leiden, Netherlands, 1988), keto-methylene pseudopeptides (Ewenson et al., J Med Chem 29:295 (1986); and Ewenson et al., in Peptides: Structure and Function (Proceedings of the 9th American Peptide Symposium), Pierce Chemical Co. Rockland, Ill., 1985), b-turn dipeptide cores (Nagai et al., Tetrahedron Lett 26:647 (1985); and Sato et al., J Chem Soc Perkin Trans 1:1231 (1986)), and b-aminoalcohols (Gordon et al., Biochem Biophys Res Commun126:419 (1985); and Dann et al., Biochem Biophys Res Commun 134:71 (1986)).

The invention is further described in the following examples, which do not limit the scope of the invention described in the claims.

### EXAMPLES

### Example 1

### G−2266A Polymorphism

The aim of this example was to identify polymorphisms in the p21 gene and examine their association with progression of proteinuria to ESRD in T1DM patients.

For this purpose, 3 kb of the promoter, all exons, and all introns were amplified and sequenced in 8 Caucasian patients with diabetic nephropathy. 24 variants were identified: 11 in the promoter, 11 in intron 1, a missence mutation (Arg31 Ser) in exon 2 and one in the intron 2. Out of 19 genotyped SNP, 15 were in strong linkage disequilibrium with a G−2266A polymorphism in the promoter (G to A variance at a position −2266 bases from the transcription start site; nucleotide corresponding to position 98 of SEQ ID NO: 1). A haplotype block including SNP in the promoter, exon1 and intron1 was found, one haplotype of which was tagged by the A allele at the G−2266A polymorphism.

221 patients with T1DM and overt proteinuria were followed for 6 years (on average), during which time ESRD developed in 51 subjects. All 221 subjects were genotyped for the G−2266A polymorphism. The risk of developing ESRD was 27% in homozygotes for G (major allele), whereas it was 8% in carriers of A (p=0.009). See Table 1.

This association was still significant in a Cox regression model after controlling for diabetes duration, HbA1c, and ACE inhibitor treatment.

Although not bound by theory, it is hypothesized that carriers of the A haplotype have decreased expression of p21 in response to a diabetic millieu, and provides protection from progression of proteinuria to ESRD.

TABLE 1
Genotype and allele distributions of the G-2266A polymorphism in the
study groups

| | |
|---|---|
| Individuals with | Individuals who |
| proteinuria | progressed to |
| at the baseline | ESRD during follow-up |

| Genotypes | n | n (%) | p-value |
|---|---|---|---|
| GG | 181 | 48 (27%) | |
| GA | 37 | 3 (8%) | |
| AA | 3 | 0 (0%) | 0.034* |
| AX | 40 | 3 (8%) | 0.009 |

*exact test

### Example 2

### G−1021A Polymorphism

Using the same methods as described in Example 1, a second variance (G to A at −1021 bases pairs from the transcription start site; nucleotide corresponding to position 98 of SEQ ID NO: 2) was identified as protective for renal disease. The A allele was present in 40% of individuals with normoalbuminuria but only 23% of individuals with proteinuria or ESRD. The frequency of carriers of A allele among controls and proteinuria or ESRD cases is shown in Table 2.

TABLE 2
Comparison of frequency of carriers of A allele among controls and cases

| | non carriers of | carriers of A allele |
|---|---|---|
| | A allele n (%) | n (%) |
| individuals with normoalbuminuria | 101 (60.48%) | 66 (39.52%) |
| (controls) | | |
| individuals with | 165 (76.74%) | 50 (23.26%) |
| proteinuria or ESRD | | |

χ² = 11.76; p = 0.0006

### Example 3

### G+1004A Polymorphism

Using the same methods as described in Example 1, a third variance was identified as protective for renal disease: G to A at +1004 base pairs from the transcription start site of p21 (nucleotide corresponding to position 103 of SEQ ID NO: 3). The A allele was present in 35% of individuals with normoalbuminuria but only 23% of individuals with proteinuria or ESRD. The frequency of carriers of the A allele among controls and proteinuria or ESRD cases is shown in Table 3.

TABLE 3
Comparison of frequency of carriers of A allele in cases and controls

| | non carriers of A allele | Carriers of A allele |
|---|---|---|
| | N (%) | N (%) |
| individuals with | 102 (64.97%) | 55 (35.03%) |
| normoalbuminuria | | |
| (controls) | | |
| individuals with proteinuria | 160 (76.56%) | 49 (23.44%) |
| or ESRD | | |

χ² = 5.92; p = 0.015

### Example 4

### Effects of p21 on Coronary Artery Disease and Mortality

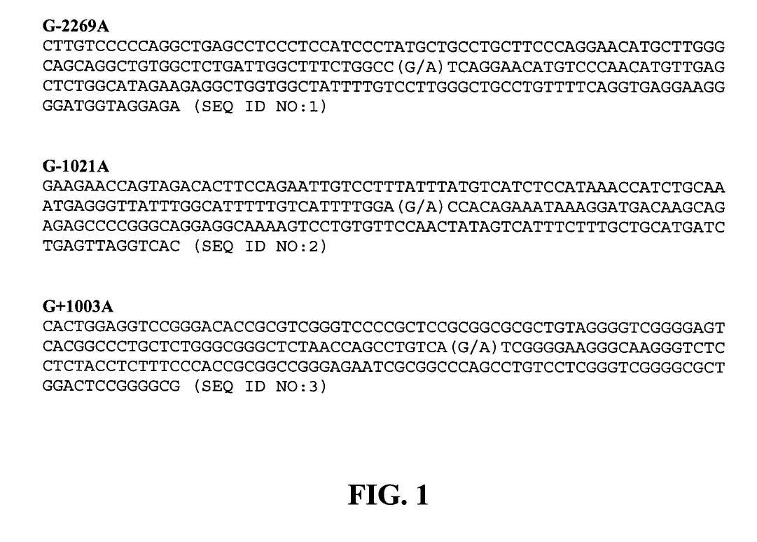

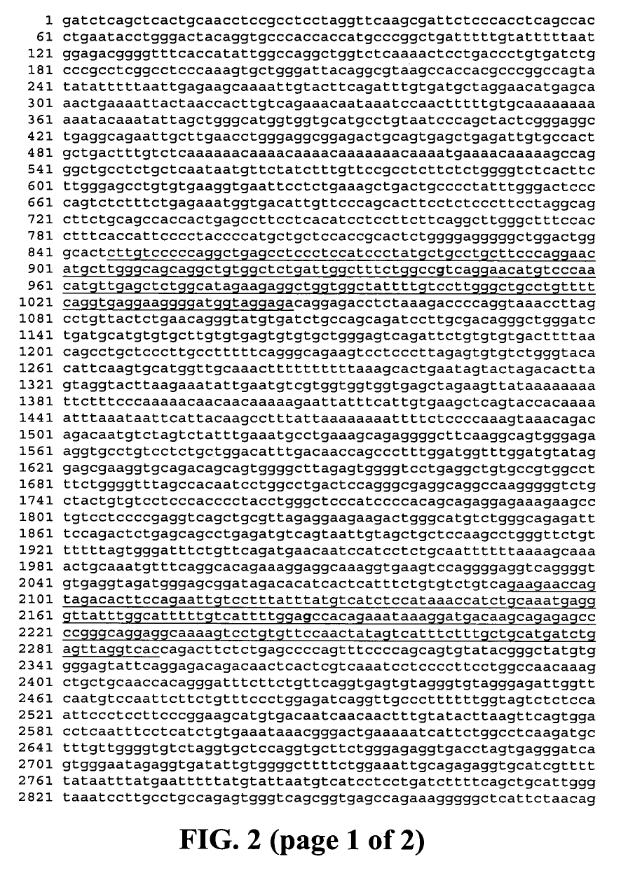

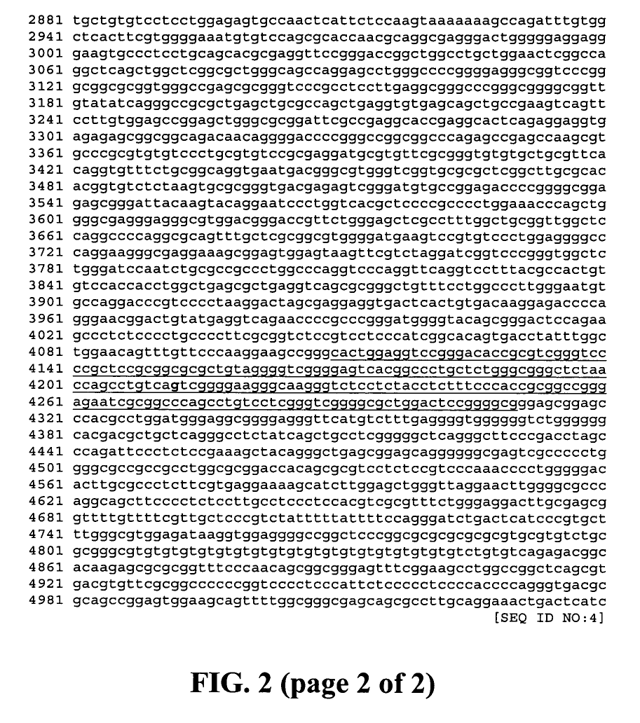

Using the data derived from the sequencing of the p21 gene in the subjects as described in Example 1, a positive correlation was found between the presence of SNPs in the p21 gene, particularly in the p21 promoter, and (a) decreased incidence of coronary artery disease (CAD) and (b) older age. With increasing age, subjects tended to have more p21 SNPs. This indicates that a decrease in p21 expression, levels or activity, e.g., caused by the presence of p21 SNPs, such as the SNPs of FIG. 1, can be protective for CAD and early mortality. Thus, in subjects who have none of the SNPs described herein, lifestyle alteration and other intervention can be used to extend lifespan and prevent early mortality.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of evaluating a subject's risk of developing renal disease or coronary artery disease (CAD), the method comprising:
determining, for one or both alleles of a p21 gene of the subject, one or more of (a) the identity of the nucleotide corresponding to position 98 of SEQ ID NO: 1, (b) the identity of the nucleotide corresponding to position 98 of SEQ ID NO: 2, and (c) the identity of the nucleotide corresponding to position 103 of SEQ ID NO: 3; and
wherein the presence of an adenine (A) at any of those positions indicates that subject has a decreased risk of developing renal disease or CAD and the presence of a guanine (G) at any of those positions indicates that the subject as an increased risk of developing renal disease or CAD.

2. The method of claim 1, wherein the determining step comprises: providing a nucleic acid sample of the subject comprising a p21 gene or fragment thereof, and detecting one or more of: (a) the identity of the nucleotide corresponding to position 98 of SEQ ID NO: 1, (b) the identity of the nucleotide corresponding to position 98 of SEQ ID NO: 2, and (c) the identity of the nucleotide corresponding to position 103 of SEQ ID NO: 3 in a nucleic acid sample of the subject.

3. The method of claim 1, wherein the determining step comprises performing a procedure selected from the group consisting of: chain terminating sequencing, restriction digestion, allele-specific polymerase reaction, single-stranded conformational polymorphism analysis, genetic bit analysis, temperature gradient gel electrophoresis, ligase chain reaction, or ligase/polymerase genetic bit analysis, allele specific hybridization, size analysis; nucleotide sequencing, 5′ nuclease digestion; primer specific extension; and oligonucleotide ligation assay.

4. The method of claim 1, wherein the subject has a family history or renal disease.

5. A probe or primer less than 500 nucleotides in length, comprising at least 10 contiguous nucleotides of SEQ ID NO:1, SEQ ID NO:2, SEQ ID NO:3, SEQ ID NO:4 or the complement of SEQ ID NO:1, SEQ ID NO:2, SEQ ID NO:3, or SEQ ID NO:4.

6. The probe or primer of claim 5, wherein the probe or primer comprises a detectable label.

7. The probe or primer of claim 5, wherein the probe or primer is attached to a solid support.

8. The probe or primer of claim 5, selected from the group consisting of:
a probe or primer that hybridizes specifically to the sequence of SEQ ID NO: 1 where position 98 is a G but not to the sequence of SEQ ID NO:1 where position 98 is an A;
a probe or primer that hybridizes specifically to the sequence of SEQ ID NO:1 where position 98 is an A but not to the sequence of SEQ ID NO:1 where position 98 is a G;
a probe or primer that hybridizes specifically to the sequence of SEQ ID NO:2 where position 98 is a G but not to the sequence of SEQ ID NO:2 where position 98 is an A;
a probe or primer that hybridizes specifically to the sequence of SEQ ID NO:2 where position 98 is an A but not to the sequence of SEQ ID NO:2 where position 98 is a G;
a probe or primer that hybridizes specifically to the sequence of SEQ ID NO:3 where position 103 is a G but not to the sequence of SEQ ID NO:3 where position 103 is an A;
a probe or primer that hybridizes specifically to the sequence of SEQ ID NO:3 where position 103 is an A but not to the sequence of SEQ ID NO:3 where position 103 is a G.

9. An array of nucleic acid molecules comprising two or more probes or primers according to claim 5.

10. An isolated fragment of a p21 gene, wherein the fragment is 5 to 200 nucleotides in length and comprises a portion of SEQ ID NO:1 comprising the nucleotide corresponding to position 98 of SEQ ID NO:1, wherein the nucleotide at position 98 is an A, T, or C.

11. An isolated fragment of a p21 gene, wherein the fragment is 5 to 200 nucleotides in length and comprises a portion of SEQ ID NO:2 comprising the nucleotide corresponding to position 98 of SEQ ID NO:2, wherein the nucleotide at position 98 is an A, T, or C.

12. An isolated fragment of a p21 gene, wherein the fragment is 5 to 200 nucleotides in length and comprises a portion of SEQ ID NO:3 comprising the nucleotide corresponding to position 103 of SEQ ID NO:3, wherein the nucleotide at position 103 is an A, T, or C.

13. A set of oligonucleotides comprising two or more of:
an oligonucleotide that hybridizes specifically to the sequence of SEQ ID NO:1 where position 98 is a G but not to the sequence of SEQ ID NO:1 where position 98 is an A;
an oligonucleotide that hybridizes specifically to the sequence of SEQ ID NO:1 where position 98 is an A but not to the sequence of SEQ ID NO:1 where position 98 is a G;
an oligonucleotide that hybridizes specifically to the sequence of SEQ ID NO:2 where position 98 is a G but not to the sequence of SEQ ID NO:2 where position 98 is an A;
an oligonucleotide that hybridizes specifically to the sequence of SEQ ID NO:2 where position 98 is an A but not to the sequence of SEQ ID NO:2 where position 98 is a G;
an oligonucleotide that hybridizes specifically to the sequence of SEQ ID NO:3 where position 103 is a G but not to the sequence of SEQ ID NO:3 where position 103 is an A; and
an oligonucleotide that hybridizes specifically to the sequence of SEQ ID NO:3 where position 103 is an A but not to the sequence of SEQ ID NO:3 where position 103 is a G.

14. An allele-specific oligonucleotide, wherein said oligonucleotide comprises a sequence complementary to a polynucleotide at a region corresponding to nucleotide position 98 of SEQ ID NO:1 of a human p21 promoter.

15. An allele-specific oligonucleotide, wherein said oligonucleotide comprises a sequence complementary to a polynucleotide at a region corresponding to nucleotide position 98 of SEQ ID NO:2 of a human p21 promoter.

16. An allele-specific oligonucleotide, wherein said oligonucleotide comprises a sequence complementary to a polynucleotide at a region corresponding to nucleotide position 103 of SEQ ID NO:3 of a human p21 promoter.

17. A kit comprising at least one probe or primer according to claim 5, and instructions for using the kit to evaluate susceptibility for a renal disorder in a subject.

18. A method of treating a subject, the method comprising identifying a subject having or at risk for renal or coronary artery disease, and administering to the subject an agent than inhibits p21 expression, levels or activity.

19. A method of identifying an agent for treatment of renal or coronary artery disease (CAD), the method comprising: identifying an agent that decreases p21 expression, levels or activity; and correlating the ability of an agent to decrease p21 expression, levels or activity, with the ability to treat CAD.

20. A method of determining if a subject is at risk for renal disease or coronary artery disease (CAD), the method comprising: evaluating the gene structure, expression, protein level or activity of p21 in the subject.

21. A method for identifying a candidate compound for treatment of renal disease or coronary artery disease (CAD), the method comprising:
providing a sample comprising a p21 polypeptide or nucleic acid;
contacting the sample with a test compound; and
evaluating an effect of the test compound on a level, expression, or activity of the p21 nucleic acid or polypeptide,
wherein a test compound that decreases a level, expression, or activity of the p21 nucleic acid or polypeptide is a candidate compound for treatment of renal disease or CAD.

22. A method for identifying a candidate therapeutic agent for treatment of renal disease or coronary artery disease (CAD), the method comprising:
providing an animal model of renal disease or CAD;
contacting the animal model with a candidate compound that decreases a level, expression, or activity of the p21 nucleic acid or polypeptide; and
evaluating an effect of the candidate compound on a parameter of the disease in the animal model;
wherein a candidate compound that improves a parameter of the disease is a candidate therapeutic agent for treatment of renal disease or CAD.

23. The method of claim 22, further comprising administering the candidate therapeutic agent to a subject having renal disease or CAD, and evaluating an effect of the candidate therapeutic agent on the disease in the subject.

24. The probe or primer of claim 5, wherein hybridization of the probe or primer allows determination of the identity of the nucleotide at one or more of (a) the nucleotide corresponding to position 98 of SEQ ID NO:1, (b) the nucleotide corresponding to position 98 of SEQ ID NO: 2, and (c) the nucleotide corresponding to position 103 of SEQ ID NO: 3.

25. The probe or primer of claim 5, wherein the probe or primer hybridizes adjacent to one or more of (a) the nucleotide corresponding to position 98 of SEQ ID NO: 1, (b) the nucleotide corresponding to position 98 of SEQ ID NO: 2, and (c) the nucleotide corresponding to position 103 of SEQ ID NO: 3.

26. The probe or primer of claim 25, wherein the probe or primer hybridizes within about 25, 50, 100, 500, 1000, 5000, or 10,000 nucleotides of (a) the nucleotide corresponding to position 98 of SEQ ID NO: 1, (b) the nucleotide corresponding to position 98 of SEQ ID NO: 2, or (c) the nucleotide corresponding to position 103 of SEQ ID NO: 3.

27. The probe or primer of claim 5, wherein the probe or primer hybridizes to a restriction fragment, wherein one or more of (a) the nucleotide corresponding to position 98 of SEQ ID NO: 1, (b) the nucleotide corresponding to position 98 of SEQ ID NO: 2, and (c) the nucleotide corresponding to position 103 of SEQ ID NO: 3 forms part of a restriction enzyme recognition site at one end of the fragment.

28. The probe or primer of claim 27, wherein the site is recognized by a restriction enzyme selected from the group consisting of HaeI, BaII, and CviJI.


SEQUENCE LISTING (4 sequences)

SEQ ID NO: 1 - DNA, 201 bp, Homo sapiens
cttgtcccccaggctgagcctccctccatccctatgctgcctgcttcccaggaacatgcttgggcagcaggctgtggctctgattggctttctggccrtcaggaacatgtcccaacatgttgagctctggcatagaagaggctggtggctattttgtccttgggctgcctgttttcaggtgaggaaggggatggtaggaga

SEQ ID NO: 2 - DNA, 201 bp, Homo sapiens
gaagaaccagtagacacttccagaattgtcctttatttatgtcatctccataaaccatctgcaaatgagggttatttggcatttttgtcattttggarccacagaaataaaggatgacaagcagagagccccgggcaggaggcaaaagtcctgtgttccaactatagtcatttctttgctgcatgatctgagttaggtcac

SEQ ID NO: 3 - DNA, 201 bp, Homo sapiens
cactggaggtccgggacaccgcgtcgggtccccgctccgcggcgcgctgtaggggtcggggagtcacggccctgctctgggcgggctctaaccagcctgtcartcggggaagggcaagggtctcctctacctctttcccaccgcggccgggagaatcgcggcccagcctgtcctcgggtcggggcgctggactccggggcg

SEQ ID NO: 4 - DNA, 5040 bp, Homo sapiens
gatctcagctcactgcaacctccgcctcctaggttcaagcgattctcccacctcagccacctgaatacctgggactacaggtgcccaccaccatgcccggctgatttttgtatttttaatggagacggggtttcaccatattggccaggctggtctcaaaactcctgaccctgtgatctgcccgcctcggcctcccaaagtgctgggattacaggcgtaagccaccacgcccggccagtatatatttttaattgagaagcaaaattgtacttcagatttgtgatgctaggaacatgagcaaactgaaaattactaaccacttgtcagaaacaataaatccaactttttgtgcaaaaaaaaaaatacaaatattagctgggcatggtggtgcatgcctgtaatcccagctactcgggaggctgaggcagaattgcttgaacctgggaggcggagactgcagtgagctgagattgtgccactgctgactttgtctcaaaaaacaaaacaaaacaaaaaaacaaaatgaaaacaaaaagccagggctgcctctgctcaataatgttctatctttgttccgcctcttctctggggtctcacttcttgggagcctgtgtgaaggtgaattcctctgaaagctgactgcccctatttgggactccccagtctctttctgagaaatggtgacattgttcccagcacttcctctcccttcctaggcagcttctgcagccaccactgagccttcctcacatcctccttcttcaggcttgggctttccacctttcaccattcccctaccccatgctgctccaccgcactctggggagggggctggactgggcactcttgtcccccaggctgagcctccctccatccctatgctgcctgcttcccaggaacatgcttgggcagcaggctgtggctctgattggctttctggccgtcaggaacatgtcccaacatgttgagctctggcatagaagaggctggtggctattttgtccttgggctgcctgttttcaggtgaggaaggggatggtaggagacaggagacctctaaagaccccaggtaaaccttagcctgttactctgaacagggtatgtgatctgccagcagatccttgcgacagggctgggatctgatgcatgtgtgcttgtgtgagtgtgtgctgggagtcagattctgtgtgtgacttttaacagcctgctcccttgcctttttcagggcagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggtgaatgacgggcgtgggtcggtgcgcgctcggcttgcgcacacggtgtctctaagtgcgcgggtgacgagagtcgggatgtgccggagaccccggggcggagagcgggattacaagtacaggaatccctggtcacgctccccgcccctggaaacccagctggggcgagggagggcgtggacgggaccgttctgggagctcgcctttggctgcggttggctccaggccccaggcgcagtttgctcgcggcgtggggatgaagtccgtgtccctggaggggcccaggaagggcgaggaaagcggagtggagtaagttcgtctaggatcggtcccgggtggctctgggatccaatctgcgccgccctggcccaggtcccaggttcaggtcctttacgccactgtgtccaccacctggctgagcgctgaggtcagcgcgggctgtttcctggcccttgggaatgtgccaggacccgtcccctaaggactagcgaggaggtgactcactgtgacaaggagaccccagggaacggactgtatgaggtcagaaccccgcccgggatggggtacagcgggactccagaagccctctcccctgccccttcgcggtctccgtcctcccatcggcacagtgacctatttggctggaacagtttgttcccaaggaagccgggcactggaggtccgggacaccgcgtcgggtccccgctccgcggcgcgctgtaggggtcggggagtcacggccctgctctgggcgggctctaaccagcctgtcagtcggggaagggcaagggtctcctctacctctttcccaccgcggccgggagaatcgcggcccagcctgtcctcgggtcggggcgctggactccggggcgggagcggagcccacgcctggatgggaggcggggagggttcatgtctttgaggggtggggggtctggggggcacgacgctgctcagggcctctatcagctgcctcgggggctcagggcttcccgacctagcccagattccctctccgaaagctacagggctgagcggagcaggggggcgagtcgccccctggggcgccgccgcctggcgcggaccacagcgcgtcctctccgtcccaaacccctgggggacacttgcgccctcttcgtgaggaaaagcatcttggagctgggttaggaacttggggcgcccaggcagcttcccctctccttgcctccctccacgtcgcgtttctgggaggacttgcgagcggttttgttttcgttgctcccgtctatttttattttccagggatctgactcatcccgtgctttgggcgtggagataaggtggaggggccggctcccggcgcgcgcgcgcgtgcgtgtctgcgcgggcgtgtgtgtgtgtgtgtgtgtgtgtgtgtgtgtgtgtgtctgtgtcagagacggcacaagagcgcgcggtttcccaacagcggcgggagtttcggaagcctggccggctcagcgtgacgtgttcgcggccccccggtcccctcccattctccccctccccaccccagggtgacgcgcagccggagtggaagcagttttggcgggcgagcagcgccttgcaggaaactgactcatc